SEQUENCE LISTING (4 sequences)

SEQ ID NO: 1 - protein, 250 aa, Bombyx mori
MIMADVAGTSNRDFRGREQRLFNSEQYNYNSSLNGEVSVWVYAYYSDGSVLVINKNSQYKVGISETFKALKEYREGQHNDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

SEQ ID NO: 2 - protein, 30 aa, Synthetic construct
MADVAGTSNRDFRGREQRLFNSEQYNYNNS

SEQ ID NO: 3 - protein, 53 aa, Synthetic construct
AFNSQRRSHTLRLLGPFQYFNFSETDRGHPLFRLPLKYPSKAIPADELIDNLH

SEQ ID NO: 4 - protein, 235 aa, Synthetic construct
GREQRLFNSEQYNYNSSLNGEVSVWVYAYYSDGSVLVINKNSQYKVGISETFKALKEYREGQHNDSYDEYEVNQSIYYPNGGDARKFHSNAKPRAIQIIFSPSVNVRTIKMAKGNAVSVPDEYLQRSHPWEATGIKYRKIKRDGEIVGYSHYFELPHEYNSISLAVSGVHKNPSSYNVGSAHNVMDVFQSCDLALRFCNRYWAELELVNHYISPNAYPYLDINNHSYGVALSNRQ

(19) United States
(12) Patent Application Publication
(10) Pub. No.: US 2026/0137625 A1
(43) Pub. Date: May 21, 2026

(54) DELIVERY OF THERAPEUTIC PROTEINS

(71) Applicant: CELL GUIDANCE SYSTEMS LIMITED, Cambridge, Cambridgeshire (GB)

(72) Inventors: Michael JONES, Royston (GB); Callum TALBOT-COOPER, Cambridge (GB); Jonathan BEST, Thaxted (GB)

(73) Assignee: CELL GUIDANCE SYSTEMS LIMITED, Cambridge, Cambridgeshire (GB)

(21) Appl. No.: 19/120,988

(22) PCT Filed: Oct. 16, 2023
(86) PCT No.: PCT/GB2023/052677

§ 371 Date: Apr. 14, 2025

(30) Foreign Application Priority Data
Oct. 17, 2022 (GB) 2215301.9

(51) Int. Cl. A61K 9/48; A61K 9/00; A61K 38/20; A61K 38/21; A61P 35/00

(52) U.S. Cl. CPC A61K 9/4825; A61K 9/0019; A61K 38/2013; A61K 38/2086; A61K 38/217; A61P 35/00

(57) ABSTRACT

The present invention relates to the treatment of diseases associated with inflammation including solid cancers. More particularly, certain methods relate to the administration (e.g., intravenously) of polyhedrin protein (PODS) crystals for the delivery of one or more therapeutic proteins. This enables targeted release of low doses of the therapeutic protein at sites of the disease.

### FIELD OF THE INVENTION

The present invention relates inter alia to the treatment of diseases associated with inflammation including solid cancers. More particularly, certain methods relate to the administration (e.g., intravenously) of polyhedrin protein (PODS) crystals for the delivery of one or more therapeutic proteins. This enables targeted release of low doses of the therapeutic protein at sites of the disease.

### BACKGROUND TO THE INVENTION

Therapeutic proteins such as cytokines and checkpoint inhibitors are beset by dose-limiting toxicity and high frequency of adverse events. For example, high-dose interleukin-2 (hIL-2), used to activate cytotoxic CD8+ T-cells (T cells) for the treatment of metastatic renal cell carcinoma (mRCC) and melanoma, suffers from wide-ranging and severe side effects including vascular leak syndrome (Choudhry et al, 2018). Although high levels of efficacy, including remission greater than 10 years, may be achieved in a small subset of patients, the high levels of toxicity experienced limits the therapeutic use of hIL-2.

IL-2 has a half-life in circulating blood of around 4 minutes (Donohue et al., 1950). This short half-life is primarily a result of active filtration by the kidneys. Consequently, high doses of IL-2 are necessary to maintain sufficient drug levels over the length of time necessary to activate T cells. hIL-2 is administered either by continuous infusion, subcutaneous or intraperitoneal injection. These administration strategies effectively increase IL-2's serum half-life to several hours. Once in circulating blood, IL-2 extravasates from the blood vessel and permeates through the tumour microenvironment (TME) to reach target cells. However, systemic therapy with immunomodulatory proteins is challenging because high protein concentrations in the serum are needed to deliver therapeutic doses to the target tissue.

PODS® (Polyhedrin Delivery System) is a natural mimetic technology based on the survival mechanism of a cypovirus that infects the larvae of Bombyx Mori, the domestic silk moth (Wendler et al., 2021). Within cypovirus-infected cells, cubic protein crystals are formed by self-assembly of a highly expressed polyhedrin protein encoded by the cypovirus. This protein crystal is used to encase and protect the progeny virion. In the PODS expression system, a secondary cargo protein is expressed in place of the viral progeny and incorporated via an immobilization tag to form PODS crystals.

Purified PODS crystals are non-brittle, durable, and transparent. PODS crystals and their cargo proteins are stable up to 37° C. in sterile solution, and across a range of pH values ranging from pH4 to pH10. Stability is lost outside this pH range and in the presence of proteases, including matrix-metalloproteases (Matsuzaki et al., 2019). These slowly degrade the crystals, releasing a stream of bioactive cargo protein over a period of 4 to 8 weeks, without burst release.

The therapeutic potential for sustained release of proteins from PODS crystals has been demonstrated in a mouse model of cartilage repair (Whitty et al., 2022) and a rat model of bone repair (Matsumoto et al., 2012) in which PODS crystals were locally administered. However, for therapy of non-localized targets, including disseminated cancers, localized drug administration is not feasible.

The utility of professional phagocytic cells to provide targeting to disease has been considered (Wendler et al., 2021). For example, exploiting monocytes and macrophages and other phagocytic immune cells as part of a molecular “Trojan horse” drug delivery strategy has previously been proposed (Cafruny et al. 1996). Monocytes are white blood cells present in circulating blood that arise from precursor cells in the bone marrow. Monocytes extravasate from blood capillaries into tissues, where they differentiate into macrophages. Monocytes infiltrate diseased and inflamed tissues in response to chemokines released by immune and cancer cells (Cassetta et al. 2018). However, efforts to date to reduce the Trojan horse idea to therapeutic practice have not been successful (Nelson et al., 2014).

It has previously been reported that in-vitro PODS crystals are efficiently taken up by phagocytic cells (Wendler et al., 2021). Moreover, following uptake, the protein cargos in the crystals are sustainably secreted in a bioactive form that is able to modulate the phenotype of adjacent heterogenous cells. However, the therapeutic efficacy and tolerability of PODS crystals containing therapeutic proteins in the treatment of non-localized targets, including disseminated cancers or other diseases, has not been demonstrated in the art.

There remains a need to develop drug delivery mechanisms that does not generate elevated serum levels of immunomodulatory proteins. There remains a need to develop drug delivery mechanisms that target diseased tissue associated with inflammation. There remains a need to develop drug delivery mechanisms that provide sustained release at the target tissue.

It is an aim of some embodiments of the present invention to mitigate some of the problems identified in the prior art.

### SUMMARY OF CERTAIN EMBODIMENTS OF THE INVENTION

The invention relates to the discovery that administration of PODS crystals (e.g., intravenously) effectively delivers therapeutic proteins to treat diseases associated with inflammation including solid and/or disseminated cancers.

Specifically, the present inventors demonstrate herein the therapeutic efficacy and tolerability of intravenously administered PODS crystals containing low doses of interleukin-2 (IL-2) in treating melanoma in vivo. The inventors also demonstrate the therapeutic benefit of IL-2, interleukin-15 (IL-15) and interferon gamma (IFN-γ) in treating renal cell carcinoma in vivo. Following this immunotherapy, cachexia, a side effect of high-dose cytokine therapy, is absent. Thus, administration of PODS crystals is shown to effectively deliver low doses of different types of therapeutic proteins whilst significantly reducing growth of different tumour types.

Advantageously, the levels of the therapeutic protein in the serum are significantly lower than needed for therapeutic effects with conventional protein therapy (e.g., when formulated for immediate release rather than utilising PODS crystals). Use of PODS crystals therefore avoid or reduce any adverse side effects associated with high-dose protein immunotherapies.

Advantageously, the PODS crystals are low-cost, easy to administer, provide sustained therapeutic protein release, high levels of stability, reduce administration frequency, have low immunogenicity, specifically target the disease and are biodegradable.

In certain embodiments, PODS crystals encapsulating the one or more therapeutic proteins are introduced directly into a subject, e.g., by intravenous injection. In such embodiments, the crystals are not contacted with one or more phagocytic cells prior to administration to the subject. Without being bound to theory, however, the PODS crystals may be internalised within phagocytic cells in vivo after their administration allowing their delivery to sites of inflammatory disease. For example, the PODS crystals may accumulate in a tumour microenvironment (TME) to reach higher levels that seen in serum. This is the opposite of what normally occurs following intravenous injection, without the combined benefit of a depot formulation and target delivery, where protein drugs perfuse from high serum levels through the tissue to the target.

In alternative embodiments, the PODS crystals are contacted (e.g., mixed) with phagocytic cells prior to injection. The efficacy of therapy may be enhanced by uptake of the PODS crystals into phagocytic cells ex-vivo before being administered to the subject. Advantageously, this may reduce the number of free PODS crystals and any off-target effects and enable higher proportions of the PODS crystals (and hence therapeutic protein) to reach the target tissue.

Whether the PODS crystals are administered directly, or pre-loaded within phagocytic cells prior to injection, the therapeutic protein(s) may be selected to modulate the phenotype of the phagocytic cells themselves. For example, macrophages exist in a spectrum of phenotypes characterised as ranging between classical M1, pro-inflammatory and alternative M2, regenerative (Cassetta et al, 2018). Tumours may subvert the behaviour of macrophages towards an M2 phenotype which is pro-tumorigenic. Without being bound to theory, the secretion of therapeutic proteins such as IFN-γ from the PODS crystals may reprogramme macrophages to an M1 phenotype. The M1 macrophages may then achieve the therapeutic effect in treating the disease.

In alternative embodiments, the therapeutic protein(s) may be selected so they do not modulate the phenotypes of the phagocytic cells. Instead, the PODS crystals may be taken up by the phagocytic cells enabling the targeted release of the therapeutic protein(s) at sites of inflammation associated with the disease. The therapeutic protein(s), delivered to the site of inflammation or macrophage accumulation, may then achieve the therapeutic effect in treating the disease.

Accordingly, the invention provides a platform technology for treating any disease associated with inflammation in a subject. In some embodiments, the disease is a cancer. For example, the cancer may be a solid cancer such as melanoma or renal cell carcinoma. The cancer may be disseminated (e.g., spread from the site of original growth to a secondary site).

In certain embodiments, the method comprises parenteral (e.g., intravenous) administration of a therapeutically effective amount of PODS crystals to the subject. Typically, the crystals are smaller than about 25 microns and/or substantially cubic in shape. For example, the modal average in crystal size is typically about 0.9 microns.

In one embodiment, the polyhedrin protein comprises the Bombyx mori cypovirus polyhedrin sequence as set forth in SEQ ID NO:1 or a variant thereof as described herein.

As further described herein, the PODS crystals encapsulate one or more therapeutic proteins. This allows the sustained release of the therapeutic protein(s) after the PODS crystals have been administered to the subject. For example, proteases may slowly degrade the crystals, leading to the sustained release of the therapeutic protein(s) at the site of inflammatory disease.

The therapeutic protein selected for use with the PODS crystals will depend on the disease being treated. Typically, the protein is an immunomodulatory protein.

In certain embodiments, phagocytic cells may take up the PODS crystals after the PODS crystals have been administered to the subject. Alternatively, the PODS crystals may be pre-loaded into phagocytic cells prior to administration.

In certain embodiments, the therapeutic protein is capable of modulating the phenotype of the phagocytic cells. For example, the therapeutic protein may be capable of stimulating M1 macrophage cell polarization. The therapeutic protein may be capable of re-programming M2 tumor-associated macrophages (TAMS), inflammatory monocytes and/or myeloid derived suppressor cells (MDSCs) into M1 macrophage cells. The modified phagocytic cells may then themselves achieve the therapeutic effect in treating the disease.

In certain embodiments, the therapeutic protein is capable of targeting a cell, extracellular vesicle or free protein in a tumour microenvironment. The therapeutic protein may stimulate immune cell activity and/or CD8+ T cells.

In certain embodiments, the therapeutic protein has a short-half life. For example, the therapeutic protein may have a serum half-life of less than about two-weeks, less than about one week, less than about 5 days, less than about 4 days, less than about 3 days, less than about 2 days, less than about 1 day, less than about 10 hours, less than about 5 hours, or about 60, about 30, about 15, about 10 or about 5 minutes, about 4 minutes or less.

In certain embodiments, the therapeutic protein is a growth factor, cytokine, checkpoint inhibitor antibody (or antibody variant such as nanobody), CD47 antibody, chemokine, endostatin, antibody, enzyme or peptide drug. In some embodiments, the therapeutic protein is a cytokine such as IL-2, IL-15, IFN-γ or the like. In certain embodiments, the cargo material is a nucleic acid or small molecule drug.

In certain embodiments, the invention provides a method of treating melanoma, wherein the method comprises administration (e.g., intravenous) of a therapeutically effective amount of PODS crystals, wherein the crystals encapsulate one or more cytokines (e.g., IL-2, IL-15 and/or IFN-γ). In such embodiments, the crystals typically encapsulate IL-2.

In certain embodiments, the invention provides a method of treating renal cell carcinoma, wherein the method comprises administration (e.g., intravenous) of a therapeutically effective amount of PODS crystals, wherein the crystals encapsulate one or more cytokines (e.g., IL-2, IL-15 and/or IFN-γ).

In certain embodiments, the therapeutic protein comprises a H1 tag sequence, as further described herein. In certain embodiments, alternative tags are used. For example, VP3, PH or any other type of suitable tag may be used.

In certain embodiments, a low dose of the therapeutic protein is administered as compared to the dose required for an equivalent therapeutic protein without the PODS crystals. The dose may depend, for example, on the type of therapeutic protein amongst other factors as further described herein.

In certain embodiments, the dose of the therapeutic protein may result in a maximum serum concentration reduced by a factor at least about 10-fold, about 20-fold, about 30-fold, about 40-fold, about 50-fold or more as compared to an equivalent amount of an unmodified therapeutic protein formulated for immediate release (e.g., intravenously administered without the PODS crystals). For example, the dose may result in a maximum serum concentration of about 400 ng per ml, about 300 ng per ml, about 200 ng per ml, about 100 ng per ml, about 80 ng per ml, about 40 ng per ml, about 20 ng per ml, about 10 ng per ml, about 8 ng per ml, about 4 ng per ml, about 2 ng per ml or less.

In certain embodiments, the dose of the therapeutic protein may be up to about 40 μg/kg of the subject's body weight. For example, the dose of the therapeutic protein may be up to about 20 μg/kg, up to about 10 μg/kg, up to about 15 μg/kg, up to about 10 μg/kg or up to about 5 μg/kg of the subject's body weight. In some embodiments, the dose of the therapeutic protein may be between about 0.1 μg/kg to about 0.2 μg/kg of the subject's body weight, between about 0.2 μg/kg to 0.8 μg/kg of the subject's body weight, or the like.

In certain embodiments, a dose of up to about 1000 crystals is administered per circulating monocyte. For example, the crystals may be in a range of about 1 to about 1000 ingested per circulating monocyte, about 2 to about 900 ingested per circulating monocyte, about 3 to about 300 ingested per circulating monocyte, about 4 to about 250 ingested per circulating monocyte, about 6 to about 100 ingested per circulating monocyte, or the like.

Any of the above-described doses may be especially effective, for example, in treating human subjects, when administering cytokines such as IL-2, IL-15 and/or IFN-γ and/or wherein the disease is a solid cancer (e.g., melanoma or renal cell carcinoma).

Typically, the PODS crystals are administered one or more times as further described herein. For example, the PODS crystals may be administered one or more times to achieve the stated maximum dose.

In certain embodiments, the crystals are administered directly to the subject e.g., by intravenous injection. In such embodiments, the crystals may be formulated in a composition which does not contain phagocytic cells. However, the crystals may be taken up by phagocytic cells within the subject's circulatory system after administration. The therapeutic protein may be secreted from the phagocytic cells intact. Typically, the therapeutic protein is sustainably released from the phagocytic cells.

In alternative embodiments, the crystals are internalised within phagocytic cells prior to administration e.g., by intravenous injection. For example, the phagocytic cells may be contacted with the crystals and cultured under conditions which induce phagocytosis. As further described herein, the phagocytic cells may be derived from peripheral blood, bone marrow, spleen or other tissue of the subject and/or expanded ex vivo before being returned to the subject.

In certain embodiments, the phagocytic cells are professional phagocytic cells. For example, the phagocytic cells may be macrophages, monocytes, neutrophils, mast cells or dendritic cells. Typically, the phagocytic cells are monocytes.

### DETAILED DESCRIPTION OF CERTAIN EMBODIMENTS OF THE INVENTION

### Brief Description of the Figures

Certain embodiments of the present invention will be described in more detail below, with reference to the accompanying Figures in which:

FIG. 1 shows ELISA measurements of accumulated IL-2 released from PCs incubated in vitro in media containing 10% serum in the presence or absence of macrophages. IL-2 was allowed to accumulate for the number of days indicated in the media before assay. Mø=Macrophages present. NC=no cargo protein in the PCs.

FIG. 2 shows ELISA measurements of serum IL-2 in treated and untreated mice. Blood samples were taken at the time intervals indicated following the final injection of PC-IL-2 into the Renal Cell Carcinoma (RENCA) and Melanoma (MEL) mice. Samples were also taken in control mice (GFP) that had received PC-GFP.

FIG. 3 shows survival and tumour size in a subcutaneous melanoma mouse model in groups of mice treated with PC-IL-2, PC-GFP and untreated (no injection). Each group initially consisted of 5 animals. A) Mean survival plot over time for each group. B) Mean tumour size (±Standard error of the mean) over time per group. C) Measurements of tumour size for individual mice over time. D) Comparison of tumour size between groups containing PCs eGFP (n=4) vs PCs IL-2 (n=5) at day 17, line represents mean value; *=p<0.05.

FIG. 4 shows Survival and tumour size in a subcutaneous RENCA mouse model in groups of mice treated with PC-IL-2, PC-IL-15, PC-IFN-γ and untreated (no injection). Each group initially consisted of 5 animals. A) Mean survival plot over time for each group. B) Mean tumour size (±Standard error of the mean) over time per group. C) Measurements of tumour size for individual mice over time. D) Comparison of tumour size between the untreated group and groups containing PCs IL-2 (n=5), PCs IL-15 (n=5) and IFN-g (n=3) at day 22, line represents mean value±SEM; *=p<0.05, **=p<0.01.

### DETAILED DESCRIPTION

The practice of embodiments of the present invention employs, unless otherwise indicated, conventional techniques of chemistry, molecular biology, pharmaceutical formulation, pharmacology and medicine, which are within the skill of those working in the art.

Most general chemistry techniques can be found in Comprehensive Heterocyclic Chemistry IF (Katritzky et al., 1996, published by Pergamon Press); Comprehensive Organic Functional Group Transformations (Katritzky et al., 1995, published by Pergamon Press); Comprehensive Organic Synthesis (Trost et al, 1991, published by Pergamon); Heterocyclic Chemistry (Joule et al. published by Chapman & Hall); Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Most general pharmaceutical formulation techniques can be found in Pharmaceutical Preformulation and Formulation (2nd Edition edited by Mark Gibson) and Pharmaceutical Excipients: Properties, Functionality and Applications in Research and Industry (edited by Otilia M Y Koo, published by Wiley).

Most general pharmacological techniques can be found in A Textbook of Clinical Pharmacology and Therapeutics (5th Edition published by Arnold Hodder).

Most general techniques on the prescribing, dispensing and administering of medicines can be found in the British National Formulary 72 (published jointly by BMJ Publishing Group Ltd and Royal Pharmaceutical Society).

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2nd ed., 2002, CRC Press; The Dictionary of Cell and Molecular Biology, 3rd ed., Academic Press; and the Oxford University Press, provide a person skilled in the art with a general dictionary of many of the terms used in this disclosure. For chemical terms, the skilled person may refer to the International Union of Pure and Applied Chemistry (IUPAC).

Units, prefixes and symbols are denoted in their Système International d′Unités (SI) accepted form. Numeric ranges are inclusive of the numbers defining the range.

### Polyhedrin Protein Crystals

Any suitable protein crystals may be used in the methods of the invention. Typically, the protein crystals are derived from viruses such as cypoviruses or bacteria such as Bacillus thuringiensis. For example, the crystals may be derived from Bombyx mori cypovirus.

The polyhedrin proteins may be generated using any suitable technique. Typically, polyhedrin crystals are generated in cells in which the polyhedrin is expressed at high levels under the control of a promoter. Co-crystals are formed when one or more therapeutic proteins are co-expressed and incorporated into the crystal. Typically, the promoters are polyhedrin promoters. Typically, the crystals are generated in insect cells. For example, the crystals may be generated using Spodoptera frugiperda 9 (Sf9) cells.

In certain embodiments, the therapeutic protein is tagged with a short peptide sequence which causes the protein to bind to the growing polyhedrin crystal. As the crystal continues to grow, the therapeutic protein becomes encased. As such, the crystals encapsulate one or more therapeutic proteins. Any suitable binding tag may be used.

In certain embodiments, the polyhedrin protein comprises the Bombyx mori cypovirus polyhedrin sequence as set forth in SEQ ID NO: 1. Alternatively, the polyhedrin protein may comprise a sequence which has at least about 70%, 80%, 85%, 90%, 95%, 96%, 97%, 98%, 99% or more homology to the sequence of SEQ ID NO: 1 based on amino acid identify over the entire sequence of SEQ ID NO: 1.

In certain embodiments, the one or more therapeutic proteins comprise a polyhedrin binding tag selected from the H1-tag sequence set forth in SEQ ID NO:2. Alternatively, the polyhedrin binding tag may comprise a sequence which has at least about 70%, 80%, 85%, 90%, 95%, 96%, 97%, 98%, 99% or more homology to the sequence of SEQ ID NO: 2 based on amino acid identify over the entire sequence of SEQ ID NO: 2.

In certain embodiments, the one or more therapeutic proteins comprise a polyhedrin binding tag selected from the VP3-tag sequence set forth in SEQ ID NO:3. Alternatively, the polyhedrin binding tag may comprise a sequence which has at least about 70%, 80%, 85%, 90%, 95%, 96%, 97%, 98%, 99% or more homology to the sequence of SEQ ID NO: 3 based on amino acid identify over the entire sequence of SEQ ID NO: 3.

In certain embodiments, the one or more therapeutic proteins comprise a polyhedrin binding tag selected from the PH-tag sequence set forth in SEQ ID NO:4. Alternatively, the polyhedrin binding tag may comprise a sequence which has at least about 70%, 80%, 85%, 90%, 95%, 96%, 97%, 98%, 99% or more homology to the sequence of SEQ ID NO: 4 based on amino acid identify over the entire sequence of SEQ ID NO: 4.

Typically, the crystals are regular arrays of polyhedrin protein assembled in a crystal lattice. The crystals may be cubic or any other shape. The crystals of the invention are typically isolated from cells, such as insect cells, which have been infected with virus, typically baculovirus or cypovirus. The crystals are typically smaller than about 25 microns, about 20 microns, about 15 microns or about 10 microns. For example, the crystals may be between about 0.1 to about 10 microns (measured as the maximum distance inside the crystal between different surfaces or the maximum length of an edge). The crystals may be treated prior to use to modify their shape and/or reduce their size. Typically, the crystals have a modal average size of about 0.9 microns.

In certain embodiments, the crystals are isolated from the cells e.g. by cell lysis, washing and centrifugation. Typically, the crystals are prepared in an aqueous suspension. For example, the concentration of the crystal suspension may be more than 10⁴ crystals per millilitre volume, typically from 10⁴ to 10⁷ crystals per millilitre volume, preferably from 5×10⁴ to 5×10⁶ or 10⁵ to 10⁶ crystals per millilitre volume.

Advantageously, the crystals are highly stable and rigid. The crystals may be attached (e.g. dried to a solid support). Typically, high levels of bioactivity are maintained after 6 months storage in protease-free solution at 37° C. Typically, the crystals are also non-toxic to the subject.

### Therapeutic Proteins

The PODS crystals may encapsulate any one or more therapeutic proteins. The therapeutic protein(s) which is selected will typically depend on the specific disease being treated.

In certain embodiments, the polyhedrin protein crystals encapsulate a single type of therapeutic protein. Alternatively, the polyhedrin protein crystals may encapsulate a combination of different therapeutic proteins. For example, the phagocytic cells may comprise at least 2, 3, 4, 5, 6, 7, 8, 9, 10 or more different therapeutic proteins.

In certain embodiments, the therapeutic proteins comprise a polyhedrin binding tag as further described herein. In embodiments where there are multiple therapeutic proteins, each may have the same polyhedrin binding tag. Alternatively, each protein may have different polyhedrin binding tags.

In certain embodiments, the therapeutic proteins are secreted from the crystals intact.

In certain embodiments, the crystals are coated with an agent, for example, protein, that delays the release of the therapeutic protein.

In certain embodiments, the one or more therapeutic proteins are capable of stimulating M1 macrophage cell polarization.

In certain embodiments, the one or more therapeutic proteins are capable of re-programming M2 tumor-associated macrophages (TAMS), or monocytes into M1 macrophage cells.

In certain embodiments, the therapeutic proteins are capable of targeting a cell, extracellular vesicle or free protein in a tumour microenvironment.

In certain embodiments, the one or more therapeutic proteins stimulate immune cell activity.

In certain embodiments, the one or more therapeutic proteins stimulate CD8+ T cells.

In certain embodiments, the one or more therapeutic proteins activate STAT1, activate Notch, promote synthesis of ROS and/or NO release, promote AKt1 kinase activity, activate NF-κB signaling, or inhibit JNK activity or CSFR1.

In certain embodiments, the one or more therapeutic proteins are growth factors, cytokines, checkpoint inhibitors, CD47 inhibitors, chemokines, endostatins, antibodies and/or variants (e.g., nanobodies), virus or virus-like particles, enzymes, nucleic acids, or peptide drugs.

In certain embodiments, the one or more checkpoint inhibitors are against programmed death protein-1 (PD-1 or CD279), PD-1 ligands PD-L1 (CD274, B7-H1) or PDL-2 (CD272, B7-DC) or cytotoxic T-lymphocyte-associated 4 (CTLA-4) or Inducible T Cell Costimulator (ICOS). For example, the checkpoint inhibitor may be Atezolizumab (Tecentriq), Avelumab (Bavencio), Dostarlizumab (Jemperli), Durvalumab (Imfinzi), Ipilumab (Yervoy), Nivolumab (Opdivo), Cemiplimab (LIbtayo), Pembrolizumab (Keytruda) or biosimilars thereof.

In certain embodiments, the one or more therapeutic proteins comprises one or more Th1-related cytokines.

In certain embodiments, the one or more cytokines are interleukins and/or interferons. For example, the cytokine may be IL-2, IL-7, IL-12, IL-15, IL-21, IFN-γ, GM-CSF, IFN-α and/or IFN-β.

In certain embodiments, the one or more therapeutic proteins have a serum half-life of less than about one week. For example, the therapeutic protein(s) may have a serum-half-life of less than about 600 minutes, about 500 minutes, about 400 minutes, about 300 minutes, about 200 minutes, about 100 minutes, about 60 minutes, about 45 minutes, about 30 minutes, about 15 minutes, about 10 minutes, about 5 minutes, about 4 minutes or less.

In certain embodiments, the one or more cytokines are engineered. For example, the cytokines may be cytokine mutants (superkines) or chimeric antibody-cytokine fusion proteins (immunokines).

In certain embodiments, the one or more cytokines are interleukin-2 (IL-2), interleukin-15 (IL-15) and/or interferon gamma (IFN-γ). Typically, such cytokines are used to treat solid cancers such as melanoma or renal cell carcinoma as further described herein.

In certain embodiments, the therapeutic proteins are recombinantly produced using a polyhedrin binding or immobilization tag as described elsewhere herein. This facilitates encapsulation of the therapeutic protein(s) during co-expression with the PODS crystals. Nucleotide sequences encoding the therapeutic protein are available through public databases (e.g., Genbank or the like). By way of non-limiting Examples, the nucleotide sequence of human IL-2 may be retrieved through Gene ID: 3558 (Genbank). The nucleotide sequence of human IL-15 may be retrieved through Gene ID: 3600 (Genbank). The nucleotide sequence of human IFN-γ may be retrieved through Gene ID: 3458 (Genbank).

### Direct Injection of PODS Crystals

In certain embodiments, the PODS crystals are administered directly to the subject. In certain embodiments, the PODS crystals are administered by parenteral injection. For example, the PODS crystals may be administered intra-arterially, intraperitoneally, intra-thecally, intraventricularly, intrasternally, intracranially, intra-muscularly, subcutaneously or the like.

In preferred embodiments, the crystals are administered by intravenous (IV) injection. The invention, relates, in part, to the surprising discovery that direct injection of the PODS crystals (e.g., without prior contact with phagocytic cells) leads to levels of therapeutic protein in serum which are much lower than would be needed for a therapeutic effect with an equivalent conventional therapy (e.g., without the PODS crystals).

After their administration, the crystals may be internalized within phagocytic cells of the subject. Advantageously, this may facilitate the delivery of the PODS crystals (and hence therapeutic proteins) to the site of the disease.

After being internalised within the phagocytic cells, the crystals are typically capable of secreting the therapeutic protein from the phagocytic cells. Typically, the therapeutic proteins are released from the crystals and retain their natural conformation and/or function. Typically, the therapeutic proteins are released through the action of proteases (e.g. matrix metalloproteases) within the cell.

In certain embodiments, the therapeutic proteins are sustainably released from the phagocytic cells. For example, the therapeutic proteins may be released from the cell to the extracellular environment. Advantageously, the therapeutic proteins may modify the behaviour of neighbouring cells that may not themselves contain any crystals. In certain embodiments, the therapeutic proteins are released from the phagocytic cells via exosomes.

In certain embodiments, the therapeutic proteins have a desired phenotypic effect upon the phagocytic cell in which they are secreted. For example, the therapeutic proteins may be capable of repolarizing the phagocytic cell (e.g. to a M1 macrophage phenotype).

Typically, the therapeutic proteins are sustainably released over a period of time that is longer than that required for the phagocytic cell to reach and/or infiltrate its intended target. For example, the therapeutic proteins may be sustainably released from the phagocytic cells over a period of at least 2 days, 5 days, 7 days, 10 days, 14 days, 20 days, 21 days, one month or more.

In certain embodiments, the phagocytic cells are professional phagocytic cells, for example, macrophages, monocytes, neutrophils, mast cells, dendritic cells or the like. Alternatively, the phagocytic cells may be non-professional cells, for example, chondrocytes, fibroblasts, lymphocytes, erythrocytes, epithelial cells or the like.

Uptake of PODS Crystals into Phagocytic Cells Prior to Injection

In alternative embodiments, the PODS crystals are contacted with phagocytic cells before being administered to the subject. For example, the phagocytic cells may be mixed with the crystals and cultured under conditions which induce phagocytosis prior to their injection.

In certain embodiments, the phagocytic cells are expanded ex vivo before being returned to the subject. Ex vivo procedures for isolating, culturing, and expanding hematopoietic stem and progenitor cells are well known in the art and further described herein. The phagocytic cells may be autologous with respect to the subject. Alternatively, the cells can be allogeneic, syngeneic or xenogeneic with respect to the subject.

In certain embodiments, the crystals are attached to a solid support prior to contact with the phagocytic cells. Examples of suitable solid supports include, but are not limited to slides, membranes and tissue culture plates. Microcarriers may be also used. The crystals may be maintained in a non-dried form and, for example, centrifuged and/or dried to a support shortly before contact with the phagocytic cells.

In alternative embodiments, the crystals are pre-mixed with the phagocytic cells. Typically, the crystals are pre-mixed with phagocytic cells for up to 12, 24, 36, 48 hours or more.

The phagocytic cells may be cultured under any conditions which induce phagocytosis. Phagocytosis of the crystals into the cells may be determined by any suitable immunohistochemistry (IHC) technique. In certain embodiments, the crystals themselves may comprise additional cargo (e.g. detectable agents such as GFP and/or luciferase) that allow phagocytosis into the cells to be verified (e.g. by fluorescent microscopy).

In certain embodiments, the phagocytic cells may be cultured in complete medium. Typically, the complete medium is supplemented with PMA. Typically, the phagocytic cells are cultured for up to 12, 24, 36, 48 hours or more. For example, the phagocytic cells may be cultured in complete medium (e.g. DMEM supplemented with 4.5 g/L D-Glucose, L-glutamine, 10% heat-inactivated FBS, 100 U/mL penicillin and 100 μg/mL Glutamax optionally supplemented with 20 ng/ml M-CSF) at a seeding density of 0.5-1.0 e6/ml at 37° C.

In certain embodiments, the phagocytic cells may be differentiated into polarizations that mimic in vivo phenotypes (e.g. a cytotoxic phenotype). For example, the culture media may be supplemented with additional immune stimulators such as LPS and/or IFN-γ to induce M1 macrophage polarization. Typically, 100 ng/ml LPS or 20 ng/ml IFN-γ is added to complete medium to induce M1 macrophage polarization after 12 hours culture in complete medium.

In certain embodiments, the phagocytic cells may be unaffected by the ingested crystals which contain a protein, such as IL-2, that targets other cells in diseased tissue.

In certain embodiments, the phagocytic cells of the invention are obtained from monocytes. For example, the phagocytic cells may be obtained from immortalized monocyte-like cell lines (e.g. THP-1 ATCC).

In certain embodiments, pre-existing phagocytic cells are isolated from body cavity lavages (alveolar, peritoneal) of resident or elicited monocytes (Zhang et al, 2008).

In certain embodiments, the phagocytic cells of the invention are obtained by isolating monocytes from blood or spleen or extracted from bone marrow. For example, the phagocytic cells may be obtained from peripheral blood mononuclear cells (PBMCs).

In certain embodiments, monocytes may be differentiated from progenitor cells by supplementing culture media with growth factors or signaling molecules such as cytokines. For example, the culture media may be supplemented with macrophage colony-stimulating factors (M-CSF). The cells may also be incubated with additional immune stimulators such as LPS and/or interferon gamma (IFN-γ). Use of such stimulators may induce different polarizations that mimic in vivo phenotypes (Mosser et al, 2008).

In certain embodiments, the phagocytic cells may comprise therapeutic proteins that do not have any effect on the phagocytic cells themselves but are secreted to affect the behaviour of other cells.

Techniques are available in the art to generate phagocytic cells in large numbers for autologous cell-based therapies. Typically, phagocytic cells such as macrophages are produced from progenitor cells. For example, such progenitor cells may be established by overexpression of Hoxb8 in media supplemented with GM-CSF (Redecke et al., 2013). This results in rapidly proliferating, cloneable cells. Removal of Hoxb8 activity allows progenitors to differentiate in phagocytic cells (e.g. macrophages).

The phagocytic cells may be isolated, substantially isolated, purified or substantially purified. The phagocytic cells are isolated or purified if they are completely free of any other components, such as culture medium, other cells or other cell types. The phagocytic cells are substantially isolated if mixed with carriers or diluents, such as culture medium, which will not interfere with their intended use.

The phagocytic cells may be isolated using any suitable technique. For example, the phagocytic cells may be isolated by leukapheresis and/or elutriation (Faradji et al, 1994; Andreeson et al. 1990). Typically, the phagocytic cells are isolated from peripheral blood of a subject as described herein. The phagocytic cells (e.g. monocytes) may then be collected from blood and isolated using a combination of leukapheresis and elutriation.

In certain embodiments, the phagocytic cells are treated ex vivo. In particular, the phagocytic cells may be loaded with polyhedrin protein crystals (themselves encapsulating one or more therapeutic proteins as described herein) and used therapeutically in the methods of the invention.

In certain embodiments, the phagocytic cells are provided in frozen aliquots and substances such as DMSO may be present to facilitate survival during freezing. Such frozen cells will typically be thawed and then placed in a buffer or medium either for maintenance or for administration.

In certain embodiments, the phagocytic cells are provided in a package. For example, the package may protect the cells from damage during collection, cell culture or transport. Suitable packages include, for example, polypropylene tubes, Teflon bags or the like.

Diseases Associated with Inflammation

The PODS crystals may be used to treat and/or prevent any disease associated with inflammation. For example, the PODS crystals may facilitate delivery of the therapeutic proteins to target a cell, extracellular vesicle and/or free protein in the tumour microenvironment. Herein, the “tumour microenvironment” is understood to include the environment around a solid cancer, including the surrounding blood vessels, immune cells, fibroblasts, signalling molecules and extracellular matrix (ECM).

In certain embodiments, the PODS crystals may encapsulate therapeutic proteins which target an immune cell, for example, a T cell or a dendritic cell. The target may also be a stromal cell or a cancer cell.

In certain embodiments, the disease is a malignant disease such as a disseminated cancer.

In certain embodiments, the PODS crystals are delivered to sites of inflammation via phagocytic cells. In such embodiments, the disease to be treated may be one in which phagocytic cells contribute and/or mediate the pathology of the disease. For example, monocytes and/or macrophages may accumulate at the site of inflammation and contribute to the disease through one or more mechanisms such as phagocytosis or release of hydrolytic enzymes and cytokines.

In certain embodiments, the PODS crystals modulate the phenotype of the phagocytic cells themselves. In such embodiments, the disease to be treated may be associated with M2 macrophage activation. Such disorders are known in the art and can include, by way of non-limiting example, Ommen's syndrome, pathogenic angiogenesis, chronic graft versus host disease (GVHD), atopic disorders, asthma, eczema, allergic rhinitis and allergies.

In certain embodiments, the disease is a cancer. The cancer may be locally advanced or metastatic. Typically, the disease is a solid cancer. For example, the PODS crystals may reduce angiogenesis, invasion, metastasis, growth and/or cell proliferation of the cancer. The subject being treated may have PD-1 or PD-L1 positive cancer cells (e.g., detected by IHC).

In certain embodiments, the PODS crystals may be used to treat cancers such as prostate cancer, breast cancer, cervical cancer, colon cancer, rectal cancer, lung cancer, ovarian cancer, testicular cancer, stomach cancer, bladder cancer, pancreatic cancer, liver cancer, kidney cancer, brain cancer, melanoma, non-melanoma skin cancer, carcinoma, bone cancer, lymphoma, leukemia, thyroid cancer, endometrial cancer, multiple myeloma, acute myeloid leukemia, neuroblastoma, glioblastoma or non-Hodgkin's lymphoma.

In certain embodiments, the carcinoma is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, lung carcinoma, small cell lung carcinoma, bladder carcinoma or epithelial carcinoma.

In certain embodiments, the PODS crystals are used to treat renal cell carcinoma. In such embodiments, the therapeutic protein may be a cytokine such as IL-2, IL-15, or IFN-γ. Alternatively, the therapeutic protein may be any other protein as described herein.

In certain embodiments, the PODS crystals are used to treat melanoma. In such embodiments, the therapeutic protein may be a cytokine such as IL-2, IL-15 or IFN-γ. Typically, the cytokine is IL-2. Alternatively, the therapeutic protein may be any other protein as described herein.

In certain embodiments, the PODS crystals comprise a checkpoint inhibitor for treatment of metastatic melanoma, renal cell carcinoma, head and neck cancer or non-small lung cancer.

In certain embodiments, the melanoma is a cutaneous, metastatic, or metastatic malignant melanoma. The melanoma may be superficial a spreading melanoma, nodular melanoma, acral lentiginous melanoma or lentigo maligna melanoma. The melanoma may be a mucosal, ocular uveal or choroidal melanoma.

In certain embodiments, the PODS crystals comprise IFNα for the treatment of resected high-risk melanoma.

In certain embodiments, the PODS crystals comprise IL-2 for treatment of metastatic renal cell carcinoma or melanoma.

### Dosages

In the methods described herein, a therapeutically effective amount of the PODS crystals is administered to the subject.

The “subject” may be murine, ovine, bovine, porcine, canine, equine, feline or any other mammal. An animal model may be used to determine the appropriate dosage for administration in humans.

Typically, the subject is a human. The subject may be an infant, juvenile or adult. Typically, the subject is suffering from the disease as further described herein.

In certain embodiments, the subject is one that has been diagnosed as having the disease. The subject may receive either prophylactic or therapeutic treatment. Treatment is considered prophylactic if administered to an individual susceptible to, or otherwise at risk of the disease. Treatment is considered therapeutic if administered to an individual suspected of having, or already suffering from the disease and/or symptom associated with the disease.

A “therapeutically effective amount” is an amount of the PODS crystals containing the therapeutic protein(s) that produces a desired therapeutic effect in the subject, such as preventing or treating the disease or alleviating symptoms associated with the disease.

The PODS crystals may be administered in an amount that will be immunologically, prophylactically and/or therapeutically effective. The quantity to be administered may depend on the subject to be treated, capacity of the subjects' immune system to respond to the antigen, degree of toxic side effects observed and the degree of response desired.

Precise amounts of the pharmaceutical composition required to be administered may depend on the judgement of the practitioner and may be peculiar to each subject. For example, the precise therapeutically effective amount is an amount of the PODS crystals that will yield the most effective results in terms of efficacy of treatment in a given subject. This amount will vary depending upon a variety of factors, including but not limited to the characteristics of the therapeutic protein (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. A person skilled in the clinical and pharmacological arts will be able to determine a therapeutically effective amount of the PODS crystals, for example, by monitoring a subject's response to administration of the crystals and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005.

In certain embodiments, the doses of the PODS crystals are guided by therapeutic doses of the conventional therapeutic protein (e.g., absent of the PODS crystals). Advantageously, use of PODS crystals allows a lower dosage of the therapeutic protein to be administered reducing toxicity in the subject. By way of example, hIL-2 therapy uses 600,000 international units (IU) per kg (Boyman et al., 2019). As further described herein, the use of PODS crystals allows administration of lower equivalent doses, determined by in-vitro levels of accumulation, of IL-2 or other therapeutic proteins (e.g., about 60,000 IU per kg, about 30,000 IU per kg, about 20,000 IU per kg, about 15,000 IU per kg, about 12,000 IU per kg or less).

In certain embodiments, the dose of the PODS crystals may deliver an amount of therapeutic protein that exhibits a reduced side effect profile as compared to a dose of the conventional therapeutic protein. For example, the PODS crystals may reduce the maximum serum concentration of the therapeutic protein, thereby reducing drug toxicity whilst maintaining drug efficacy. Such toxicities may include, for example, hypotension, acute renal insufficiency, respiratory failure, neuropsychiatric symptoms and the like.

As used herein, “serum concentration” refers to the concentration of the therapeutic protein in the blood serum of the subject. It is understood that the maximum serum concentration of a therapeutic protein may vary from subject to subject, due, for example, to variability with respect to metabolism of the therapeutic protein(s). Due to this variability, the amount necessary to constitute a therapeutically effective amount of therapeutic protein may vary from subject to subject.

As used herein, the “maximum serum concentration” (Cmax) is the peak serum concentration that a therapeutic agent achieves in a specified area of the body after the therapeutic agent has been administered and before the administration of any second dose.

In certain embodiments, the therapeutic effective amount of the PODS crystals may deliver a dose of the therapeutic protein that results in a maximum serum concentration reduced by at least about 10-fold, about 20-fold, about 30-fold, about 40-fold, about 50-fold, about 100-fold, about 150-fold, about 200-fold, about 300-fold or more as compared to an equivalent amount of the therapeutic protein administered by intravenous bolus injection without the PODS crystals. Typically, the dose of the therapeutic protein results in a maximum serum concentration reduced by at least about 50-fold as compared to an equivalent amount of the therapeutic protein administered without the PODS crystals.

By way of non-limiting example, the therapeutic protein may be IL-2, IL-15 or IFN-γ and the maximum serum concentration may be reduced by at least about 50-fold or more as compared to an equivalent amount of the therapeutic protein administered without the PODS crystals.

In certain embodiments, the therapeutic effective amount of the PODS crystals may deliver a dose of the therapeutic protein that results in a maximum serum concentration of about 400 ng per ml, about 300 ng per ml, about 200 ng per ml, about 100 ng per ml, about 80 ng per ml, about 40 ng per ml, about 20 ng per ml, about 10 ng per ml, about 8 ng per ml, about 4 ng per ml, about 2 ng per ml or less. Typically, the therapeutic effective amount of the PODS crystals may deliver a dose of the therapeutic protein that results in a maximum serum concentration of about 8 ng per ml or less.

By way of non-limiting example, the therapeutic protein may be IL-2, IL-15 or IFN-γ and the therapeutic effective amount of the PODS crystals may deliver a dose of the therapeutic protein that results in a maximum serum concentration of between about 2.0 to 8.0 ng per ml.

In certain embodiments, the therapeutic effective amount of the PODS crystals may deliver a dose of the therapeutic protein that results in a peak serum load of about 40 μg/kg, about 20 μg/kg, about 10 μg/kg, about 5 μg/kg, about 4 μg/kg, about 3 μg/kg, about 2 μg/kg, about 1 μg/kg, about 0.8 μg/kg, about 0.6 μg/kg, about 0.4 μg/kg, about 0.2 μg/kg or less of the subject's body weight. Typically, the therapeutic effective amount of the PODS crystals may deliver a dose of the therapeutic protein that results in a peak load of about 0.8 μg/kg or less of the subject's body weight.

By way of non-limiting example, the therapeutic protein may be IL-2, IL-15 or IFN-γ and the therapeutic effective amount of the PODS crystals may deliver a dose of the therapeutic protein that results in a peak load of between about 0.2 to about 0.8 μg/kg of the subject's body weight.

In certain embodiments, the therapeutic effective amount of the PODS crystals may be a dose of between about 1×10⁶ to 1×10¹⁰ crystals per kg of the subject's body weight. Typically, the therapeutic effective amount of the PODS crystals may be a dose of between about 2×10⁹ to 8×10⁹ crystals per kg of the subject's body weight when directly injected intravenously, or lower when ingested ex-vivo.

By way of non-limiting example, the therapeutic protein may be IL-2, IL-15 or IFN-γ and the therapeutic effective amount of the PODS crystals may be a dose of between about 2×10⁹ to 8×10⁹ crystals per kg of the subject's body weight.

In certain embodiments, the therapeutic effective amount of the PODS crystals, considering that not all PODS crystals will be taken up by monocytes, may result in a maximal burden of about 1 to 1000 crystals ingested per circulating monocyte. Typically, the therapeutic effective amount of the PODS crystals may result in a maximal burden of about 5 to about 80 crystals.

By way of non-limiting example, the therapeutic protein may be IL-2, IL-15 or IFN-γ and the therapeutic effective amount of the PODS crystals may deliver a dose of the therapeutic protein that results in a maximal burden of about 6 to 75 crystals ingested per circulating monocyte.

In certain embodiments, the time it takes for the blood concentration of a therapeutic protein to fall by one half is referred to as the “serum half-life” of a substance, irrespective of the factors (e.g. plasma clearance, kidney filtration, absorbance by the tissue) which cause the decrease of concentration.

In certain embodiments, the dose of the PODS crystals may be administered as a single dose or as multiple doses (e.g., 2, 3, 4, 5, 6 or more doses). For example, more than one dose (e.g., bolus or infusion) may be administered over a time period of about a month.

In some embodiments, the PODS crystals are administered weekly or two or three times a week. In some embodiments, the PODS crystals are administered daily. In some embodiments, the PODS crystals are administered two or more times daily (e.g., 2, 3, 4, 5, or more times).

In some embodiments, the subject is administered multiple doses (e.g., over the course of 2 days, 3 days, 4 days, 5 days, 6 days, 1 week, 2 weeks, 3 week or month).

The PODS crystals may be administered to the subject over any suitable time period. For example, the PODS crystals may be administered to the subject over a time period from about 30 seconds to about 48 hours. The PODS crystals may be administered to the subject over about 30 minutes or less. Alternatively, the PODS crystals may be administered to the subject over about 30 minutes or more.

In certain embodiments, the therapeutic protein is administered by intravenous bolus injection. An intravenous bolus injection may be a single, large dose of the therapeutic protein over a short period of time. Typically, the intravenous bolus injection is administered in about 30 minutes or less.

In alternative embodiments, the therapeutic protein is administered by intravenous infusion. An intravenous infusion may be the continuous administration of the therapeutic protein over a time period longer than 30 minutes.

In certain embodiments, the PODS crystals are administered to the subject in combination with one or more other therapeutic agents. By combination, it is meant that the therapies may be administered simultaneously, in a combined or separate form, to the subject. The therapies may be administered separately or sequentially to the subject as part of the same therapeutic regimen. For example, the PODS crystals of the invention may be used in combination with another therapy intended to treat the disease associated with inflammation in the subject.

In embodiments where the PODS crystals are used in combination with one or more other therapeutic agents, the additional therapeutic agents may be administered by the same route (e.g., intravenously) or by a different route.

In certain embodiments, the PODS crystals are combined with treatment with one or more chemotherapeutic agents. Typically, the chemotherapeutic agents are selected from Dacarbazine (DTIC), Temozolomide, Nab-paclitaxel, Paclitaxel, Carmustine (BCNU), Cisplatin, Carboplatin, Vinblastine, or any combination thereof.

In certain embodiments, the PODS crystals are combined with treatment with one or checkpoint inhibitors as described herein. For example, the PODS crystals may comprise any one or more therapeutic proteins as described herein other than a checkpoint inhibitor. In such embodiments, the additional therapeutic agent may be a checkpoint inhibitor against PD-1, PDL-1, ICOS or CTLA-4 (e.g., Atezolizumab, Avelumab, Dostarlizumab, Durvalumab, Ipilumab, Nivolumab, Cemiplimab, Pembrolizumab or the like).

### Pharmaceutical Compositions

In certain embodiments, the PODS crystals are formulated as a pharmaceutical composition. The pharmaceutical composition may be used to treat any disease described herein.

In certain embodiments, the pharmaceutical composition does not contain any phagocytic cells.

In alternative embodiments, the pharmaceutical composition comprises phagocytic cells.

Formulation of the PODS crystals with standard pharmaceutically acceptable carriers and/or excipients may be performed using routine methods. The exact nature of a formulation will depend upon several factors, including the therapeutic proteins to be administered. Suitable types of formulation are fully described in Remington's Pharmaceutical Sciences, 19th Edition, Mack Publishing Company, Eastern Pennsylvania, USA.

In certain embodiments, the pharmaceutical composition is formulated for parenteral (e.g., intravenous) delivery. For example, the PODS crystals may be formulated with at least one pharmaceutically acceptable excipient, adjuvant or carrier.

Typically, the PDOS crystals are administered intravenously. Methods and formulations for intravenous administration of therapeutic proteins are well known in the art. For intravenous administration, suitable carriers include physiological saline, bacteriostatic water, Cremophor EL™ (BASF, Parsippany, N.J.) or phosphate buffered saline (PBS). Aptly, the composition is sterile and fluid to the extent that easy syringeability exists. Aptly, it is stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminium monostearate and gelatin.

The PODS crystals may be administered intravenously and/or intra-peritoneally to reach the circulatory system of the subject. The PODS crystals may be administered as a bolus or by continuous infusion, and suitable dosages are described herein. Injectables can be prepared either as liquid solutions (or suspensions) or solid forms suitable for solution in (or suspension in) liquid prior to injection.

The invention further provides any pharmaceutical composition or PODS crystals as described herein for use in a method of treating any disease as described herein, wherein the method comprises administration (e.g., intravenous) of a therapeutically effective amount of the crystals.

The invention further provides use of any pharmaceutical composition or PODS crystals as described herein for the manufacture of a medicament for treating any disease as described herein.

The invention also provides expression cassettes or vectors comprising a nucleic acid encoding the PODS crystals and/or therapeutic proteins (e.g., IL-2, IL-15 and/or IFN-γ) optionally under the control of any promoter (e.g., polyhedrin promoter) as described herein. Typically, the therapeutic proteins also comprise one or more polyhedrin-binding tag sequences as further described herein. Nucleic acid sequences encoding the therapeutic proteins as described herein are readily available through public online databases (e.g., Genbank or the like).

The invention also provides transformed cells, transduced cells, host cells and the like containing such expression cassettes, vectors or PODS crystals. For example, the invention also provides a population comprising more than about 1×10⁷ phagocytic cells, wherein the phagocytic cells comprise PODS crystals encapsulating one or more therapeutic proteins (e.g., IL-2, IL-15 and/or IFN-γ) as described herein. Typically, the population is autologous.

### EXAMPLES

In the following, the invention will be explained in more detail by means of non-limiting examples of specific embodiments. In the example experiments, standard reagents and buffers free from contamination are used.

### Example 1—Materials and Methods

### PODS Crystal Production

PODS crystals (PCs) were essentially prepared as previously described (Matsumoto et al., 2012, Nishishita et al., 2011, herein incorporated by reference).

Briefly, to generate empty (CP-H) polyhedra, Spodoptera frugiperda IPLB-SF21-AE cells (Sf cells) were inoculated with recombinant baculovirus AcCP-H29/PDI expressing BmCPV polyhedrin, in which BmCPV polyhedrin is expressed under the control of the baculovirus polyhedrin promoter. For production of IL-2, IL-15 or IFN-γ polyhedra, Sf cells were infected with AcCP-H29 also expressing recombinant IL-2, IL-15 or IFN-γ fused with the H1 tag. The infected cells were cultured for 10 days at 27° C. and then the cells were harvested in a conical tube by centrifugation. The cell pellet was resuspended in phosphate-buffered saline (PBS; pH 7.2) and treated with an ultrasonic homogenizer at 6% power for 30 sec. The cell homogenate was centrifuged at 1500×g at 4° C. and the supernatant was removed. These treatments were repeated and the purification was complete. The polyhedron suspension was adjusted to the same density (5×10⁴ cubes per microliter volume) and stored at 4° C. in distilled water containing 100 units/ml penicillin and 100 μg/ml streptomycin.

### Cell Culture and Differentiation

Monocyte suspension cells (THP-1, Public Health England Culture Collection) were cultured and differentiated as previously described (Wendler et al, 2021).

Briefly, the cells were cultured undifferentiated in RPMI-1640 (A10491, Gibco) supplemented with 10% BCS (30-2030, ATCC) (complete medium). For M0 differentiation, THP-1 cells were centrifuged and the conditioned media were replaced with fresh complete medium supplemented with 100 ng/ml phorbol 12-myristate-13-acetate (PMA, Sigma P8139). After 48 h, M0 macrophages were further differentiated into M1 or M2 macrophages.

Briefly, M0 differentiation medium was removed and adherent M0 cells were washed twice with serum-free medium. For further differentiation, complete medium supplemented with either 100 ng/ml LPS and 20 ng/ml IFN-γ (M1) or 20 ng/ml IL-4 and 20 ng/ml IL-13 (M2) was added to adherent M0 cells and then incubated for up to a further 48 h. A375 cells (ATCC) were cultured in DMEM (41966, Gibco) supplemented with 10% FBS (F7524, SIGMA). Cells were seeded at a density of 24 cell/cm² and passaged every 3 days. TF-1 cells (ATCC) were cultured in RPMI-1640 supplemented with 2 ng/ml GM-CSF and 10% FBS. Cells were seeded at a density of 25 cells/ml and passaged twice a week.

Measurement of the Release of IL-2 from PODS IL-2, with and without Macrophages

THP-1 cells were seeded at a density of 2×10⁵ cells/ml in 96-well plates and differentiated into M0 macrophages. M0 cells were then incubated with 1×10⁵, 2×10⁵, or 4×10⁵ PODS IL-6 per ml for 24 h in complete medium (resulting in 5, 10, or 20 PODS/cell). Additionally, the same number of PCs containing IL-2 (PC-IL-2) were added to the wells of a 96-well plate and spun down at 3000×g for 25 min. Cells were then washed twice with PBS, and fresh complete medium was added. After incubation, the medium was collected at 0, 1, 2, 3 and 6 days and subsequently tested by IL-2 ELISA (R&D Systems) according to the manufacturer's protocol.

### Cancer Cell Culture and Generation of Tumours

The B16-F10 melanoma cell line was obtained from ATCC (CRL-6475™). Cells were expanded in Dulbecco's Modified Eagle's medium (Corning®) containing 10% fetal bovine serum. The RENCA cell line was obtained from ATCC (CRL-2947) and cultured as above.

All animal procedures were conducted in compliance with the Stanford Administrative Panel for Laboratory Animal Care (APLAC). For the melanoma model, 6- to 8-week-old female C57/b6 mice were purchased from Jackson laboratories. 1×10⁵ B16-F10 cells in 100 μl of PBS were injected subcutaneously into the shaved lateral flank of mice anesthetised by inhalation of isoflurane. For the renal cell carcinoma (RCC) model. 6- to 8-week-old female BALB/C mice were purchased from Jackson laboratories. 2.5×10⁵ RECNA cells were injected subcutaneously as above.

### PODS Administration in Melanoma Model

Fifteen C57/b6 mice (Jackson Labs) were divided into three groups of five mice in which each mouse received controls of either no injection, 4×10⁷ PCs containing enhanced green fluorescent protein (PC-GFP), or 4×10⁷ PC-IL-2 in 200 μL of PBS. Intravenous injections were given on days 5, 7, 13, and 19 days after tumour inoculation (total 16×10⁷ PCs per mouse).

### PODS Administration in the Renal Cell Carcinoma Model

Twenty BALB/C mice (Jackson Labs) were divided into four groups of five mice in which each mouse received controls of either 1×10⁷ PC-IL-2, PC-IL-15, or PC-IFN-γ in 200 μL of PBS. Intravenous injections were given on days 7, 10, 16, and 23 days after tumour inoculation (total 4×10⁷ PCs per mouse). The remaining group of five mice were untreated controls.

### ELISA Measurements of IL-2 in Serum

30 μL blood samples were collected at 4-, 24- and 48-hours intervals from 3 BALB/C mice carrying RCC tumours receiving IL-2 therapy following the final dose and tested by IL-2 ELISA (R&D Systems) according to the manufacturer's protocol.

### Tumour Measurement

Blinded measurements of tumour length and width were performed using external callipers. Tumour volume (mm³) was calculated by the modified ellipsoidal formula: V=½ (Length×Width²). Animals were sacrificed if their tumour had reached a volume greater than 1,000 mm³, a single dimension greater than 17 mm, or if ulceration was observed.

### Statistics

Survival rates were depicted using a Kaplan-Meier curve. Tumour volume measurements were plotted and analysed using Prism 9.4.1 (GraphPad Software, LLC). Two-way ANOVAs were performed comparing the different treatment groups with respect to time. Individual data points at day 17 were also isolated for the PC-GFP and PC-IL-2 groups and compared using an unpaired student's t test. Similarly, in the RCC study, the individual data points and groups at day 22 were isolated and compared using a one-way ANOVA with post-hoc Dunnett's t-test conducted to determine any differences with the control group.

### Example 2—In Vitro IL-2 Levels

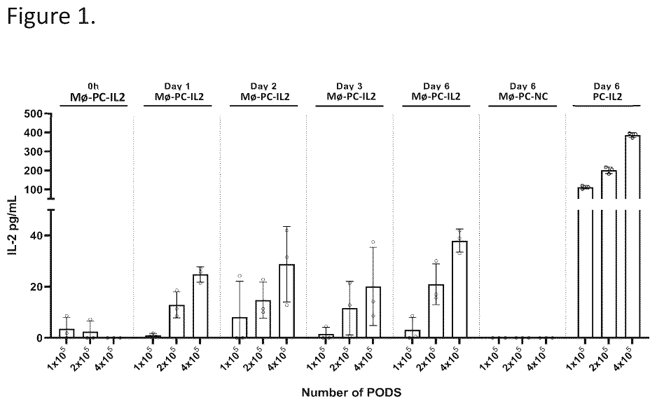

PCs provide sustained release of cargo proteins. The amount of protein that accumulates depends on the balance of release and degradation rates. To provide a basic framework for dosing in the in-vivo assays, in-vitro accumulation from PCs containing IL-2 which were incubated in serum in the presence or absence of THP-1 cell-derived macrophages was measured. Macrophages were allowed 24 hours to take up the PCs. The amount of IL-2 that accumulated in the culture media was measured using an ELISA assay at 0, 1, 2, 3 and 6 days after addition of 10% serum which provided a source of proteases to break down the PCs liberating the IL-2 cargo. The amount of IL-2 measured was dependent on the number of PODS added (FIG. 1). The amount that accumulated in the media peaked by day 1 and remained relatively constant until day 6. There was a direct relationship between the number of PODS and the amount of secreted IL-2 detected by the ELISA assay. Where IL-6 release was measured from phagocytosed PCs, the presence of macrophages, which phagocytosed the PCs, significantly reduced the amount of cargo protein accumulating in the media. In the absence of macrophages, (measured only on day 6) the amount of IL-2 that accumulated was about 10-fold higher.

### Example 3—Determining Dosing

To reduce drug toxicity, serum concentrations of IL-2 were limited to significantly below amounts used for hIL-2 melanoma and mRCC therapy. hIL-2 therapy uses 600,000 international units (IU) per kg (Boyman et al., 2019). 1 mg of IL-2 contains approximately 16 million IU making 600,000 IU equivalent to 37.5 μg/kg. In the clinic, bolus injected patients receive 3 injections per day for 5 days, or as long as they can tolerate. Since residual IL-2 will be available on the second and subsequent injections, peak load is likely to be higher than 37.5 μg/kg.

The dosing regimen was determined based on the above in-vitro release data, mouse weight and published estimates of blood volume and monocyte numbers in mice (O'Connell et al., 2015). For the melanoma study, the mice were tail vein injected with 4×10⁷ PCs per injection. Each mouse was scheduled to receive four injections (a total of 16×10⁷ PCs). Based on the levels of accumulation seen in-vitro following ingestion of PCs by macrophages, the maximal amount of IL-2 released would be 16 ng. For a typical 20 g mouse with 2 ml blood volume, this total has the maximum potential to generate a bio-available load of 0.8 μg/kg (about 50-fold lower than the typical hIL-2 concentrations of 37.5 μg/kg) or 8 ng per ml of serum with a maximal burden of 25-75 crystals/circulating monocyte.

The RCC mice received a reduced dose of 1×10⁷ PCs per injection and a total of four doses (a total of 4×10⁷ PCs). This total has the maximum potential to generate 0.2 μg/kg or 2 ng per ml of serum with a maximal burden of 6-19 crystals/circulating monocyte.

### Example 4—Melanoma Response to PC-IL-2

Fifteen C57/b6 mice were subcutaneously inoculated with B16-F10 melanoma cells. These mice were divided into three groups of five mice each. The first control group received no treatment, the second control group received PODS containing green fluorescent protein (GFP) and the final group received PC-IL-2.

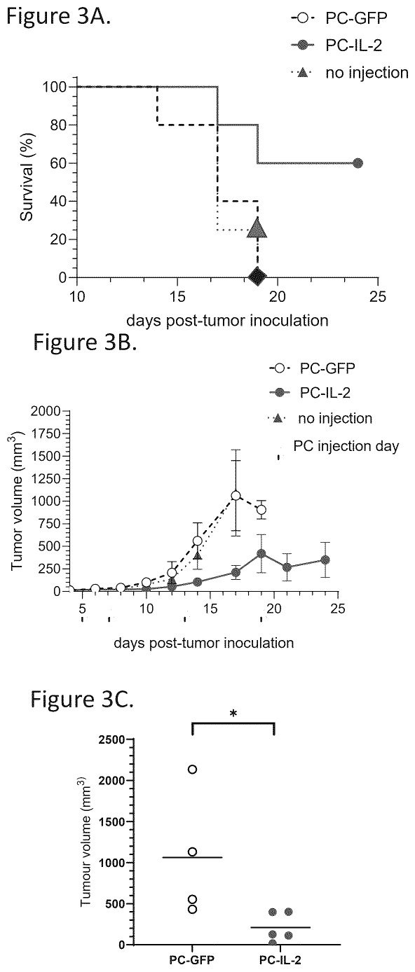

Tumours were measured, and observations of health were made, at two-to-three-day intervals. Mice were euthanized when the tumour volume exceeded 1000 mm³ or if the skin above the tumour became ulcerated. All 15 mice appeared to be generally healthy with no signs of cachexia throughout the study. The tumours in the mice in the two control groups (GFP and no injection) grew faster than tumours in the PC-IL-2 treatment group (FIG. 3). Consistent with low levels of immunogenicity and bioactivity previously reported for PCs lacking bioactive cargo proteins (Matsumoto et al, 2014, Matsumoto et al., 2015), the mice receiving PC-GFP did not have any apparent differences in overall health compared with the no-treatment group.

Since the tumour volume had exceeded 1000 mm³, the first control mouse was sacrificed 14 days after melanoma cell inoculation. Survival is plotted in FIG. 3A. By day 19, all ten control mice had been sacrificed. In contrast, the first mouse in the treatment group was sacrificed on day 17 due to skin ulceration around the tumour site and 3 of the treatment group mice survived to day 25 when the study was terminated. In two of the surviving mice, the tumour volume was stable and remained below 200 mm³. A two-way ANOVA comparing tumour size in the PC-GFP and no injection groups (FIG. 3B) was conducted using a mixed-effect model with matching time point values. There was an effect of time (F=14.31, p=0.003), but no effect of treatment (F=0.1027, p=0.76). Thus, the presence of the PCs containing the inactive GFP cargo protein did not influence the tumour growth. In contrast, a two-way ANOVA comparing the PC-GFP and PC-IL-2 treatment on melanoma tumour volume (FIG. 3B) confirmed that there an effect of time (F=6.43, p=0.005) and an effect of treatment (F=9.264, p=0.016). When combined with observations of the longitudinal data (FIGS. 3A and B), this strongly indicated that the PC-IL-2 was arresting the growth of the tumour and prolonging the survival of these mice. Due to the reduction in the numbers of mice in the control group towards the end of the study, a meaningful post-hoc analysis could not be conducted. However, to determine if the reduction in tumour volume with PC-IL-2 treatment showed a statistically significant effect compared to the PC-GFP group at later time points, a paired t-test was conducted on the data points at day 17 (FIG. 3C). This represented the latest time point with the sufficient mice available for statistical comparison. The unpaired, two-tailed test revealed a significant difference between the two groups (t=2.41, p=0.046). Although an isolated time point, this result also supports a conclusion that administration of PC-IL-2 results in the arrest of tumour growth.

In addition to treating melanoma, high dose IL-2 has regulatory approval for the treatment of metastatic renal cell carcinoma. The utility of PC-IL-2 in reducing tumour size in a mouse model of RCC using RENCA cells administered subcutaneously was therefore evaluated. In addition, the utility of the PC-IL-15 and PC-IFN-γ was explored. These are cytokines that have been reported to be effective against a range of cancers (Matsumoto et al., 2015, Overwijk et al., 2021) albeit with challenges associated with delivering an effective dose to the TME. The design of the RCC study was similar to the melanoma study, but the PC-GFP control was not used and a lower dose of each PC type was used. For each treated mouse, a dose of 1×10⁷ PCs containing a single cytokine cargo was administered a total of four times on days 5, 8, 14, and 20 days after RENCA cell inoculation for a total of 4×10⁷ PCs per mouse. This was 25% of the dose used in the melanoma study. As RENCA tumours were expected to be slower growing than the melanoma, less frequent observations of the mice and their tumours at twice weekly intervals were made. In fact, tumour growth rate was similar to the melanomas.

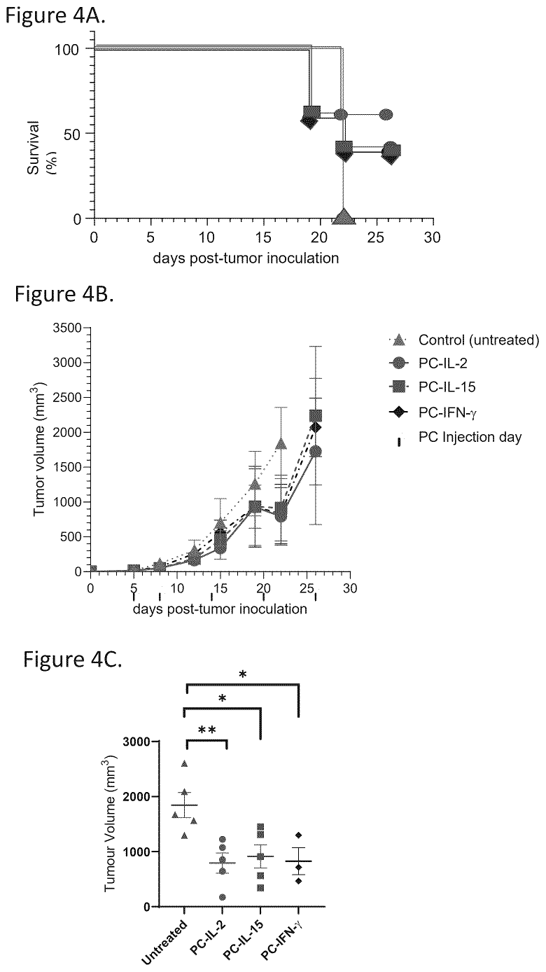

Even with the lower drug dose, RENCA tumours in all three treatment groups had longer survival (FIG. 4A) and showed slower growth (FIG. 4B) than those in the untreated group. Two-way ANOVAs were performed comparing tumour size in the different treatment groups with the control group (FIG. 4B). Due to the absence of data points for the control group at day 26, this time point was excluded from the analysis. A two-way ANOVA comparing the control group with PC-IL-2 revealed that there was an effect of time (F=55.62, p<0.0001) and treatment (F=5.63, p<0.05). Equally, there was an effect of treatment when compared with PC-IL-15 (F=6.56, p=0.014). As with the Melanoma study, the reduction in the numbers of mice in the control group did not allow a meaningful post-hoc analysis. Therefore, to determine if the reduction in tumour volume with the treatment groups showed a statistically significant effect compared to the control group, a one-way ANOVA was conducted on the data points at day 22 (FIG. 4C). This represented the latest time point with the sufficient mice for statistical analysis. The one-way ANOVA revealed a significant effect of treatment (F=5.68, p=0.009), with post-hoc analysis compared to the control being significant for all treatment groups (PC-IL-2, p=0.008; PC-IL-15, p=0.018; PC-IFNg, p=0.024). There was no significant difference between the treatment groups. Although an isolated time point, this result supports the observation that administration of each cytokine results in a reduction of tumour growth.

### Example 5—Measurement of In Vivo IL-2 Levels

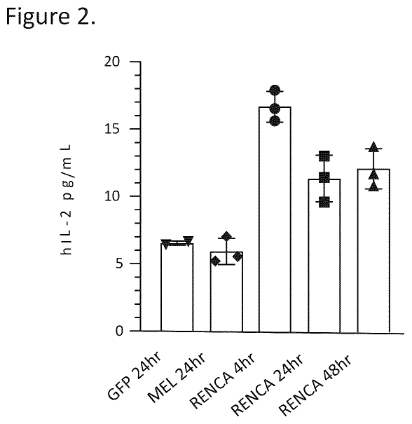

As well as demonstrating that cytokines delivered by PCs is sufficient to reduce tumour growth, whether serum concentrations were maintained at low levels was evaluated. Since the conditions in-vivo are very different to in-vitro, it was expected that kidney filtering would maintain low IL-2 serum levels. To measure the amount of IL-2 that actually accumulates in mouse sera, 30 μl blood samples were taken from three mice that were receiving PC-IL-2 therapy. For melanoma mice, samples were taken at 24 hours following the final dose. Samples were also taken in control mice that had received PC-GFP. For RENCA mice, samples were taken at 4, 24 and 48 hours following the final PC-IL-2 dose. An ELISA assay was used to measure the amount of free IL-2 that accumulated in the sera. Measurements of IL-2 were low, possibly below the limits of assay sensitivity, for GFP and IL-2 melanoma mice. In the IL-2 treated RENCA mice, levels were measured in a range between 9 μg/ml and 17 μg/ml (FIG. 2). These values are markedly below the potential maximum levels of 2 ng per ml calculated based on in-vitro release rates. This is consistent with constant clearance of released IL-2 in serum by kidney filtration preventing higher levels of IL-2 accumulation.

### Discussion

Addressing toxicity is a high priority for drug developers. For example, second-generation IL-2 drugs offering an improved therapeutic window are in development. These work by mechanisms including increased stability, improved specificity, modulating receptor binding characteristics, and tumour targeting (Overwijk et al., 2021, Ball et al., 2022). Some of these novel delivery modalities are in clinical trials.

Whilst a perfect protein delivery system is likely to remain elusive, the mechanism provided by PCs has many key attributes that make it particularly attractive: Manufacture of PCs is simple, scalable and low cost. Sustained release is achieved over several weeks without burst release. Moreover, PCs are well tolerated in mouse models, are biodegradable, and can be targeted to disease sites using phagocytic cells. This combination of attributes generates a unique ability in PCs to significantly skew the biodistribution of protein drugs to reduce toxicity and increase efficacy. PCs work by slowly releasing their protein cargo, which then accumulates for as long as secretion rates are higher than elimination rates. The concentration of cargo proteins that accumulate in the space surrounding each PC will depend on a number of factors. These include (1) the protease-dependent release rate, (2) the inherent stability of the cargo protein, (3) the concentration of proteases in the extracellular space that will degrade released protein, (4) the presence of molecules that may bind and stabilize the released protein, (5) metabolism by cells and (6) flushing provided by circulating fluids and subsequent filtering that may remove the released protein. As expected, the measurements of IL-2 that accumulated in-vitro, with only proteolytic degradation as a clearance mechanism, were significantly higher than those measured in serum in-vivo.

The sustained release of cargo proteins from PCs allows localized concentrations of the cargo protein to accumulate. Since the TME is largely isolated from the filtering effects of the kidney, it can be expected that protein drugs will accumulate in the TME to higher levels than seen in serum. This is the opposite of what normally occurs following intravenous injection, without the combined benefits of a depot formulation and targeted delivery, where protein drugs perfuse from high serum levels through to markedly lower levels the tissue to the target.

The doses of PCs used in the studies conducted here were not guided by prior dose finding studies. Instead, they were guided by therapeutic doses for conventional recombinant IL-2 used in the clinic combined with data from the in-vitro release studies. The aim was to treat the tumours with doses of IL-2 that are significantly below those used in hIL-2 studies to reduce toxic side effects. It is possible that higher doses may achieve greater efficacy whilst maintaining sufficiently low levels of toxicity to avoid cachexia and other treatment side effects.

The amount of IL-2 measured in sera was very low. Unexpectedly, the serum levels in the RENCA mice which had received a lower dose, were higher than in the melanoma mice. Whilst the tumours may have an impact on serum levels, the observation may also be associated with the different genetic background of the two mouse strains with C57/b6 mice used for the melanoma study and BALB/C mice used for the RCC study. Further studies will be needed to understand any underlying mechanism. Importantly, any adverse effects in any of the mice treated with PCs was not observed. In particular, cachexia was absent.

It has been previously demonstrated that PCs are efficiently taken up by monocytes in-vitro. In-vivo, this mechanism is unlikely to be as efficient: A large proportion of the PCs will lodge directly in other tissues or be filtered, for example by the Kupfer cells of the liver. These off-target PCs will also secrete cargo and, if secretion rates are sufficiently high, cargo proteins secreted from PCs located away from the tumour may also reach the tumour. It is not clear to what extent the therapeutic effect is mediated by the release of IL-2 from PCs that have lodged in the tumour versus IL-2 released by PCs elsewhere in the body. However, given the very low serum IL-2 levels measured, it is unlikely that free IL-2 transported to the tumour, rather than released within the tumour from PCs, was primarily responsible for observed efficacy. Cargo proteins released from PCs lodged in non-tumour sites may accumulate and cause toxicity.

Rather than exceeding limitations set for tumour size, many of the treatment group mice that were euthanized during the study had smaller tumours with ulceration of the skin above the tumour. Ulceration is often associated with inflammation, and the propensity of the treated tumours to ulcerate more frequently than the untreated tumours may reflect higher levels of inflammation around the tumour associated with the treatment.

Amongst professional phagocytic cells, monocytes (and the macrophages they differentiate into) are the most likely mediators of PC delivery to tumours. However, it should be noted that neutrophils, mast cells, and dendritic cells also phagocytose efficiently and infiltrate inflamed tissue and cancers (Lim et al., 2017). These cells may also contribute to transport of the PCs to the tumour target.

As an alternative to direct injection of PCs, efficiency of therapy could be enhanced by introducing PCs to phagocytic immune cells ex-vivo. This could be achieved using monocytes or other phagocytic cells collected by apheresis or by harvesting monocyte progenitor cells from the bone marrow and expanding them in-vitro. After a period of incubation to allow uptake of the PCs, the loaded phagocytic cells would be returned to the patient. Compared to other autologous cell-based therapies, such as CAR-T therapy, such a procedure would require very simple manipulation of the cells and would be expected to reduce the number of free PCs that are filtered by Kupfer cells or lodge in non-target tissue enabling higher proportions of PC-loaded monocytes to reach the target tissue. As well as reducing toxicity, this may further increase efficacy.

hIL-2 provides a therapeutic effect by increasing T cell activity within the TME. As well as delivering protein to target adjacent heterologous cells within the TME, PCs could be used to directly modulate the phenotype of the macrophage itself. Macrophages exist in a spectrum of phenotypes broadly characterised as ranging between classical M1, pro-inflammatory and alternative M2, regenerative phenotypes (Cassetta et al, 2019) Tumours subvert the behaviour of macrophages towards a pro-tumorigenic M2 phenotype. Protein drugs such as IFN-γ can contribute to reprogramming of macrophages to an M1 phenotype. The therapeutic effect seen here for IFN-γ may have been achieved, at least in part, through this mechanism.

In addition to cytokines, many other protein drugs suffer from poor PKPD profiles. For example, checkpoint inhibitors, presently at the vanguard of immuno-oncology, produce adverse effect in about 40% of patients (Morgado et al., 2020). These can be severe enough to halt treatment or cause fatality. The drug delivery mechanism demonstrated here, using PCs to deliver a protein therapeutic to cancer, could readily be applied to other protein drugs and other diseases where inflammation actively recruits phagocytic immune cells. Protein drugs that could be delivered by PCs include other growth factors, chemokines, antibodies and antibody variants (e.g. nanobodies), viruses and virus-like particles, enzymes (e.g. hyaluronidase), peptide drugs and nucleic acids. Moreover, combination therapies with protein/protein and protein/non-protein drugs, such as chemotherapeutics, as well as in combination with surgery may provide higher levels of therapeutic efficacy.

### REFERENCES

1. Yang Y, Lundqvist A. Immunomodulatory Effects of IL-2 and IL-15; Implications for Cancer Immunotherapy. Cancers. 2020; 12 (12): 3586. doi: 10.3390/cancers12123586
2. Morgado M, Plácido A, Morgado S, Roque F. Management of the Adverse Effects of Immune Checkpoint Inhibitors. Vaccines. 2020; 8 (4): 575. doi: 10.3390/vaccines8040575
3. Choudhry H, Helmi N, Abdulaal W H, et al. Prospects of IL-2 in Cancer Immunotherapy. Stockinger H, ed. BioMed Res Int. 2018; 2018:9056173. doi: 10.1155/2018/9056173
4. Donohue J H, Rosenberg S A. The fate of interleukin-2 after in vivo administration. J Immunol Baltim Md 1950. 1983; 130 (5): 2203-2208.
5. Wendler A, James N, Jones M H, Pernstich C. Phagocytosed Polyhedrin-Cytokine Cocrystal Nanoparticles Provide Sustained Secretion of Bioactive Cytokines from Macrophages. BioDesign Res. 2021; 2021. doi: 10.34133/2021/9816485
6. Matsuzaki Y, Maruta R, Takaki K, et al. Sustained Neurotrophin Release from Protein Nanoparticles Mediated by Matrix Metalloproteinases Induces the Alignment and Differentiation of Nerve Cells. Biomolecules. 2019; 9 (10): 510. doi: 10.3390/biom9100510
7. Sustained delivery of the bone morphogenetic proteins BMP-2 and BMP-7 for cartilage repair and regeneration in osteoarthritis|Elsevier Enhanced Reader. doi: 10.1016/j.ocarto.2022.100240
8. Matsumoto G, Ueda T, Shimoyama J, et al. Bone regeneration by polyhedral microcrystals from silkworm virus. Sci Rep. 2012; 2:935. doi: 10.1038/srep00935
9. Cafruny W A, Bradley S E. Trojan Horse macrophages: studies with the murine lactate dehydrogenase-elevating virus and implications for sexually transmitted virus infection. J Gen Virol. 1996; 77 (12): 3005-3012. doi: 10.1099/0022-1317-77-12-3005
10. Nelson D, Fisher S, Robinson B. The “Trojan Horse” Approach to Tumor Immunotherapy: Targeting the Tumor Microenvironment. J Immunol Res. 2014; 2014. doi: 10.1155/2014/789069
11. Cassetta L, Pollard J W. Targeting macrophages: therapeutic approaches in cancer. Nat Rev Drug Discov. 2018; 17 (12): 887-904. doi: 10.1038/nrd.2018.169
12. Paul D, Achouri S, Yoon Y Z, Herre J, Bryant C E, Cicuta P. Phagocytosis Dynamics Depends on Target Shape. Biophys J. 2013; 105 (5): 1143-1150. doi: 10.1016/j.bpj.2013.07.036
13. Nishishita N, ljiri H, Takenaka C, et al. The use of leukemia inhibitory factor immobilized on virus-derived polyhedra to support the proliferation of mouse embryonic and induced pluripotent stem cells. Biomaterials. 2011; 32 (14): 3555-3563. doi: 10.1016/j.biomaterials.2010.12.063
14. Boyman O, Arenas-Ramirez N. Development of a novel class of interleukin-2 immunotherapies for metastatic cancer. Swiss Med Wkly. 2019; 149: w14697. doi: 10.4414/smw.2019.14697
15. O'Connell K E, Mikkola A M, Stepanek A M, et al. Practical Murine Hematopathology: A Comparative Review and Implications for Research. Comp Med. 2015; 65 (2): 96-113.
16. Matsumoto G, Hirohata R, Hayashi K, et al. Control of angiogenesis by VEGF and endostatin-encapsulated protein microcrystals and inhibition of tumor angiogenesis. Biomaterials. 2014; 35 (4): 1326-1333. doi: 10.1016/j.biomaterials.2013.10.051
17. Matsumoto G, Ueda T, Sugita Y, et al. Polyhedral microcrystals encapsulating bone morphogenetic protein 2 improve healing in the alveolar ridge. J Biomater Appl. 2015; 30 (2): 193-200. doi: 10.1177/0885328215575763
18. Overwijk W W, Tagliaferri M A, Zalevsky J. Engineering IL-2 to Give New Life to T Cell Immunotherapy. Annu Rev Med. 2021; 72 (1): 281-311. doi: 10.1146/annurev-med-073118-011031
19. Ball K, Bruin G, Escandón E, et al. Characterizing the Pharmacokinetics and Biodistribution of Therapeutic Proteins: An Industry White Paper. Drug Metab Dispos. 2022; 50 (6): 858-866. doi: 10.1124/dmd. 121.000463
20. Lim J J, Grinstein S, Roth Z. Diversity and Versatility of Phagocytosis: Roles in Innate Immunity, Front Cell Infect Microbiol. 2017; 7. Tissue Remodeling, and Homeostasis. doi: 10.3389/fcimb.2017.00191

All publications, patents, patent applications and GenBank accession numbers cited herein are hereby expressly incorporated by reference for all purposes.

1. A method of treating a disease associated with inflammation in a subject, wherein the method comprises administration of a therapeutically effective amount of polyhedrin protein (PODS) crystals to the subject, wherein the crystals encapsulate one or more therapeutic proteins.

2. The method of claim 1, wherein the PODS crystals are administered intravenously.

3. The method of claim 2, wherein the therapeutically effective amount is:
(i) a dose of the therapeutic protein that results in a maximum serum concentration reduced by a factor of at least about 10-fold as compared to an equivalent amount of an unmodified therapeutic protein intravenously administered without the PODS crystals;
(ii) a dose that results in a maximum serum concentration of about 40 ng per ml;
(iii) a dose of therapeutic protein that generates total serum load up to about 4 μg/kg of the subject's body weight; and/or
(iv) a dose of up to about 100 crystals ingested per circulating monocyte.

4. The method of claim 1, wherein
the crystals are not contacted with one or more phagocytic cells prior to administration to the subject.

5. The method of claim 1, wherein the crystals are contacted with one or more phagocytic cells prior to administration to the subject, optionally wherein:
(i) the phagocytic cells are contacted with the crystals and cultured under conditions which induce phagocytosis; and/or
(ii) the phagocytic cells are derived from peripheral blood of the subject and/or expanded ex vivo before being returned to the subject.

6. The method of claim 5, wherein:
(i) the crystals are capable of secreting the therapeutic protein from the phagocytic cells intact; and/or
(ii) the proteins are capable of being sustainably released from the phagocytic cells.

7. The method of claim 5, wherein the phagocytic cells are professional phagocytic cells, optionally wherein the professional phagocytic cell is a macrophage, monocyte, neutrophil, mast cell or dendritic cell.

8. The method of claim 1, wherein the disease associated with inflammation is a cancer, wherein optionally the cancer is solid, wherein further optionally the cancer is melanoma or renal cell carcinoma.

9. (canceled)

10. (canceled)

11. The method of claim 1, wherein the subject is a human.

12. The method of claim 1, wherein the crystals are smaller than about 25 microns and/or substantially cubic in shape.

13. The method of claim 1, wherein:
(i) the polyhedrin protein comprises the Bombyx mori cypovirus polyhedrin sequence as set forth in SEQ ID NO: 1; or
(ii) the polyhedrin protein comprises a sequence which has at least 70% homology to the sequence of SEQ ID NO: 1 based on amino acid identity over the entire sequence of SEQ ID NO: 1.

14. The method of claim 1, wherein the therapeutic protein comprises one or more polyhedrin-binding tag sequences selected from:
(i) the H1-tag sequence as set forth in SEQ ID NO: 2;
(ii) the VP3-tag sequence as set forth in SEQ ID NO: 3;
(iii) the PH tag sequence as set forth in SEQ ID NO: 4; or
(iv) a sequence which has at least 70% homology to the sequence of SEQ ID NO: 2, 3 or 4 based on amino acid identity over the entire sequence of SEQ ID NO: 2, 3 or 4 respectively.

15. The method of claim 1, wherein the therapeutic protein is capable of targeting a cell, extracellular vesicle or free protein in a tumour microenvironment.

16. The method of claim 15, wherein:
(i) the therapeutic protein stimulates immune cell activity; or
(ii) the therapeutic protein stimulates CD8+ T cells.

17. The method of claim 16, wherein:
(i) the therapeutic protein is capable of stimulating M1 macrophage cell polarization; and/or
(ii) the therapeutic protein is capable of re-programming M2 tumor-associated macrophages (TAMS), inflammatory monocytes and/or myeloid derived suppressor cells (MDSCs) into M1 macrophage cells.

18. The method of claim 1, wherein the therapeutic protein has a serum half-life of less than about a week, wherein optionally the therapeutic protein has a serum half-life of less than about 5 hours.

19. (canceled)

20. The method of claim 1, wherein the therapeutic protein is a growth factor, cytokine, checkpoint inhibitor (e.g., antibody against PD, PD-L1, PD-L2, ICOS, or CTLA4), CD47, chemokine, endostatin, antibody and/or variant (e.g., nanobody), virus or virus-like particle, enzyme, peptide drug or nucleic acid, wherein optionally the cytokine is an interleukin and/or interferon, wherein further optionally the cytokine is interleukin-2 (IL-2), interleukin-15 (IL-15) and/or interferon gamma (IFN-γ).

21. (canceled)

22. (canceled)

23. PODS crystals encapsulating one or more therapeutic proteins for use in a method of treating a disease associated with inflammation in a subject, wherein the method comprises administration of a therapeutically effective amount of the crystals.

24. PODS crystals for use in a method of claim 23.

25. A pharmaceutical composition comprising PODS crystals, wherein the crystals encapsulate one or more therapeutic proteins and the pharmaceutical composition is formulated for intravenous delivery.
